(19) United States
(12) Patent Application Publication
(10) Pub. No.: US 2022/0361817 A1
(43) Pub. Date: Nov. 17, 2022

(54) Joint Sensing

(71) Applicant: McLaren Applied Technologies Limited, Woking (GB)

(72) Inventors: Nicholas H. Reddall, London (GB); Matthias Verstraete, Chaam (NL); Toni Sokura, Helsinki (FI)

(21) Appl. No.: 17/754,983

(22) PCT Filed: Oct. 15, 2020
(86) PCT No.: PCT/IB2020/059720

§ 371 Date: Apr. 18, 2022

(30) Foreign Application Priority Data
Oct. 18, 2019 (GB) 1915135.6

(51) Int. Cl. A61B 5/00; A61B 5/11; A61B 5/107

(52) U.S. Cl. CPC A61B 5/683; A61B 5/1121; A61B 5/1071; A61B 5/6828; A61B 5/4528; A61B 5/6842; A61B 5/1114; A61B 5/0024; A61B 5/6844; A61B 2560/0223

(57) ABSTRACT

A method of calibrating a pair of body mounted sensors, the method comprising the steps of: (a) in a baseline position of a joint to be measured, determining a first offset between a measured joint angle and an angle between pair of sensors, one mounted on each side of the joint to be measured, so as to calibrate the sensors; (b) after at least one of the sensors has been removed and reapplied, placing the joint back into the baseline position such that the sensors are in a second configuration relative to each other; and (c) determining a second offset between the measured knee angle and an angle between the pair of sensors in the second configuration in order to recalibrate the sensors such that, in each of the first and second configurations, the same joint angle for the baseline position is reported.

### CROSS-REFERENCE TO RELATED APPLICATIONS

This application claims the benefit of priority from United Kingdom patent application no. 1915135.6, filed Oct. 18, 2019, which is incorporated by reference herein in its entirety.

### DETAILED DESCRIPTION

This invention relates to a method for calibrating sensors to compensate for misalignment, and a system for mounting sensors to a body to reduce misalignment, typically of the sensors relative to the body.

There is a growing popularity for devices that measure movement. These sensing devices could be in the form of wearable devices that measure movement of a user, a smartphone that is carried by the user to measure movement of the user or moveable devices that can generally sense movement, for instance video game controllers or sensors attached to industrial equipment. In particular, wearable devices can be utilised to track motion of a human or other animal and, in particular can be used to monitor the motion of a specific joint.

These moveable sensing devices may include a satellite positioning sensor which can sense the location of the device, and one or more motion sensors which sense motion and/or orientation of the device. These motion sensors may include one or more of an accelerometer, a gyroscope, a magnetometer, a compass and a barometer.

When using a wearable device, it may be necessary for the device to be used for extended periods of time such as a month or more so as to build up data which changes only slowly over time. This means that any sensing device which is used will likely need to be removed for any number of reasons including, but not limited to, the need to recharge a power supply on the device, the desire to clean the sensor so as to remove a build of dirt and grime or spillages thereon, or to wash the part of the person or animal on which the sensor is mounted.

Whilst a simple method of mounting a wearable device could include one or more ties, straps or belts or other attachment systems which permit simple and easy removal, such devices can be uncomfortable for a user on whom the device is mounted.

Furthermore, removal of the sensor from the user and then a subsequent replacement or remounting of the sensor provides significant opportunity for the sensor to be replaced in a different location to the previous mounting of the sensor, and this can lead to problems and inconsistencies in the recorded data which potentially renders some or all of the data unusable. This is particularly true when two or more sensors operate together to provide data related to the relative movement of the sensors.

Thus, it would be desirable for there to be improvements in how wearable sensors can be mounted and operated.

According to the present invention, there is provided a method of calibrating a pair of body mounted sensors, the method comprising the steps of: (a) in a baseline position of a joint to be measured, determining a first offset between a measured joint angle and an angle between pair of sensors, one mounted on each side of the joint to be measured, so as to calibrate the sensors; (b) after at least one of the sensors has been removed and reapplied, placing the joint back into the baseline position such that the sensors are in a second configuration relative to each other; and (c) determining a second offset between the measured knee angle and an angle between the pair of sensors in the second configuration in order to recalibrate the sensors such that, in each of the first and second configurations, the same joint angle for the baseline position is reported.

The pair of sensors may communicate such that the angle between the sensors is determined by one of the sensors.

The method may further comprise, prior to (a), the step of measuring the joint angle by using a goniometer.

The recalibration may be carried out as part of a sensor activation process.

The step of measuring the baseline position may include measuring a joint angle between the respective portions of the joint, which may be using a goniometer. The measured joint angle may a pitch angle and/or a roll angle.

The method may further comprise the step of moving the joint to the baseline position which is preferably a joint full extension position.

Preferably reapplication of the removed sensor is carried out at substantially the same position was previously placed.

The method may further comprise the step of identifying the axis of movement of the joint.

The method may further comprise the step of applying sensors, one on each side of a joint. The method may further comprise the step of, prior to applying the sensors, marking the sensor locations on each side of the joint.

The present invention also provides a system for recording angular position changes in a joint, the system comprising: a pair of sensors, each sensor being placed, in use, on a respective side of a joint, each sensor including a data transmission device for providing data relating to the orientation of the sensor; a data storage device for receiving data from one or more of the sensors, the data relating to the orientation of one or both sensors; and a control system configured to recognise when a sensor has been removed from the joint and to require a recalibration of the alignment of the sensors prior to recording a subsequent data set.

The present invention further provides a system for mounting a removable sensor on an animal body for a time period, the system comprising: a first mount having an adhesive layer on one face for application to the surface of the animal body for a first subset of the time period; and a second mount which acts to removably fix a sensor to the first mount for a second subset of the time period, the second subset being shorter than the first.

The second mount may be a bidirectional fixing.

The second mount may comprise:
(i) a first temporary fixing system for permitting the second mount to be fixed to the first mount for the second subset of the time period; and
(ii) a second temporary fixing system for permitting a sensor to be fixed to the second mount.

Multiple second mounts may be provided, typically sufficient to allow the sensor to be repeatedly mounted to the first mount within the first subset of the time period.

Multiple first mounts may be provided to permit the first mount to be replaced after the first subset of the time period.

The second mount may include one or more of: an adhesive, a hard clip, a soft pocket, press fit fittings, directional hook and loop fasteners (Velcro®) or a magnet.

The first mount may include at least one visual indicator section through which a respective mark on the animal body can be seen to assist with alignment of a replacement first mount. Two or more visual indicator sections may be provided.

The first mount may include a multi-layer structure, preferably having layers including MED 2171 H, polyurethane film and MED 5062 A.

The second mount may have adhesive on both faces. The second mount may include a layer formed from MED 6361U.

The second mount may be in two parts, a first part being for attachment to the first mount and the second part for attachment to the sensor, such that the fixing joins the first and second parts together.

The present invention also provides a method as described according to any combination of the above features, wherein one or more of the sensors is mounted to the body using a system as described according to any combination of the above features.

The present invention will now be described by way of example with reference to the accompanying drawings. In the drawings:

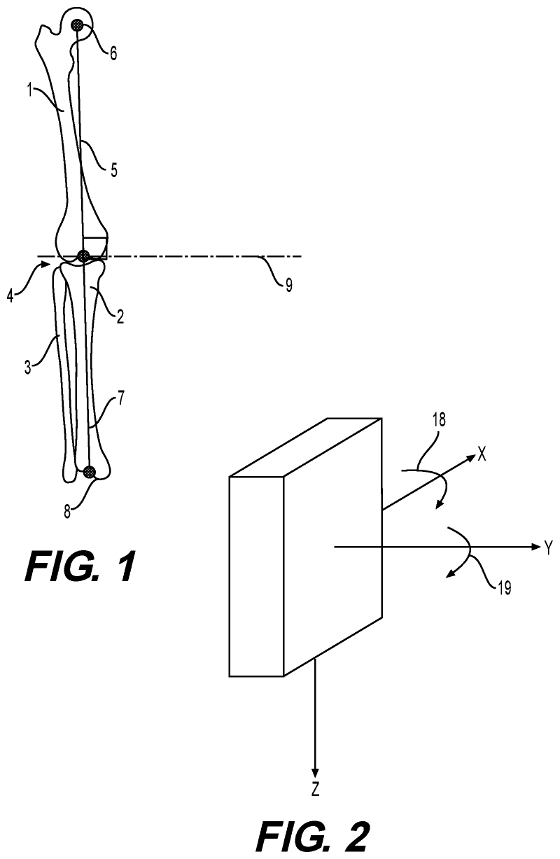

FIG. 1 shows a diagram of a joint;

FIG. 2 shows a direction reference frame for a joint;

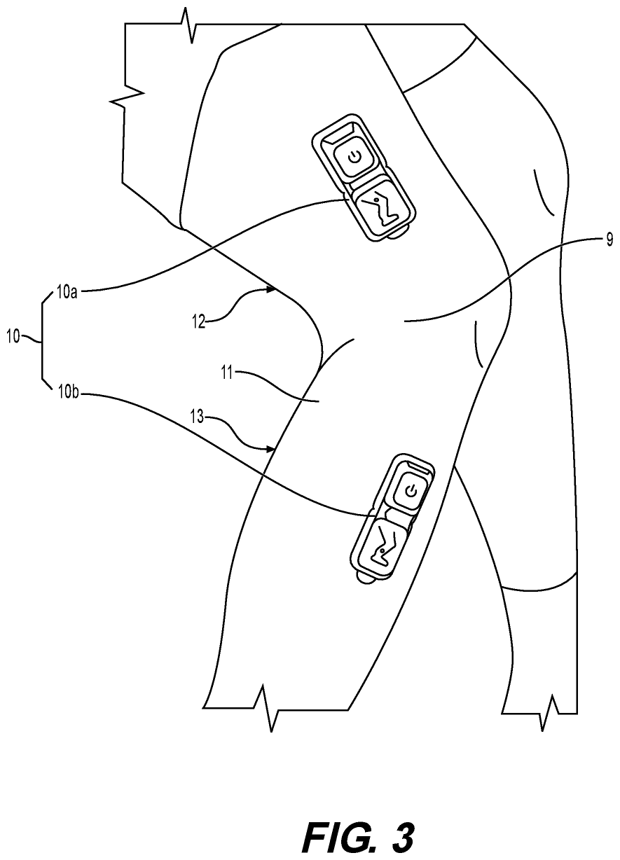

FIG. 3 shows a pair of sensors fitted either side of a joint in accordance with some implementations;

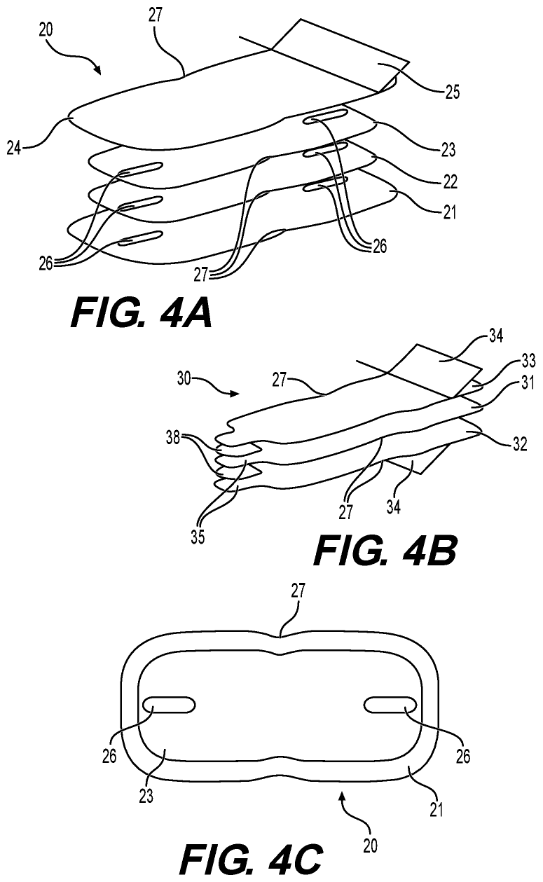

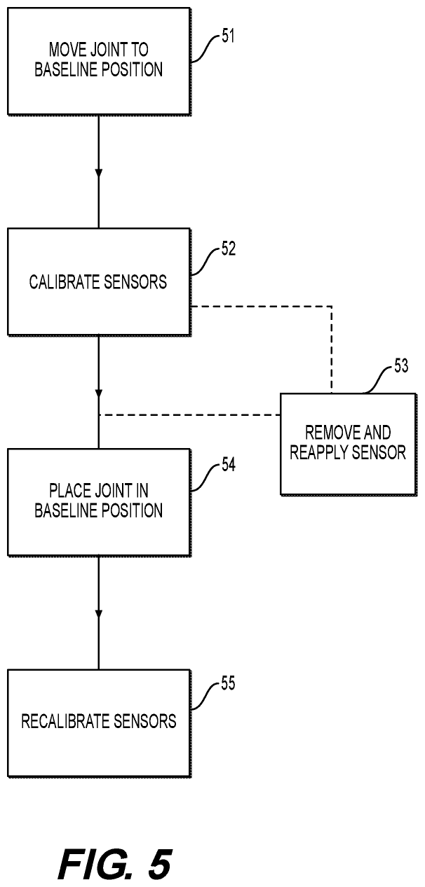

FIG. 4(a) to (c) shows a sensor mounting system;

FIG. 5 shows a schematic method for operating the sensors and/or calibrating them;

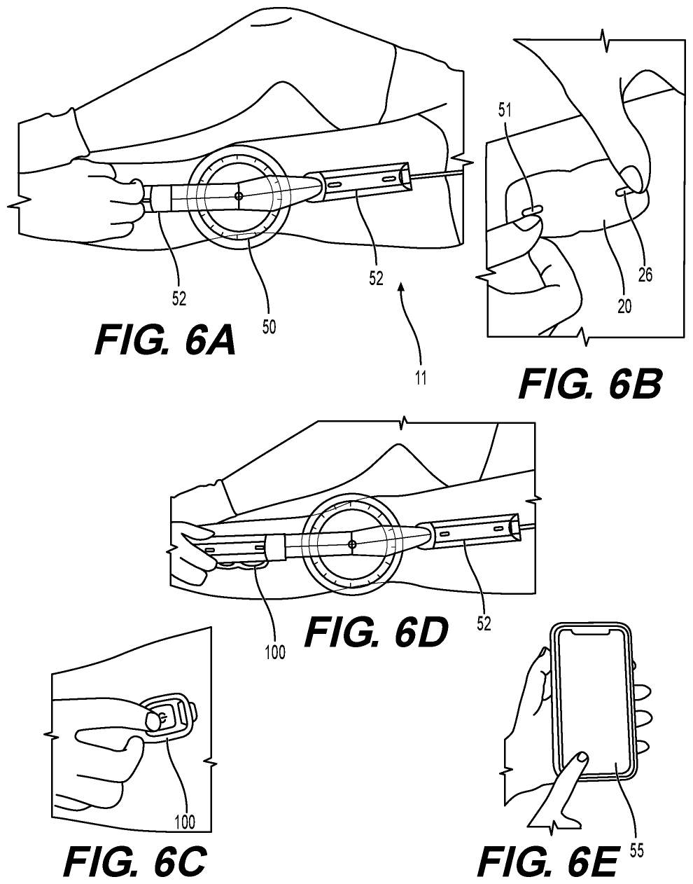

FIG. 6(a) to (e) shows a method of calibrating the sensors; and

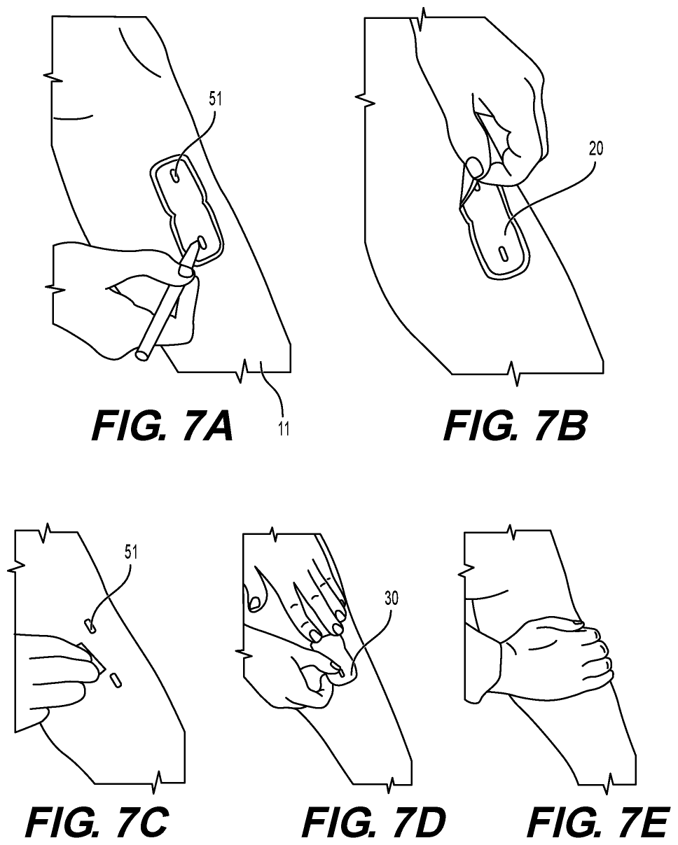

FIG. 7(a) to (e) shows a method of using the sensor mounting system.

Whilst this specification describes specific examples of the use of sensors in relation to a knee joint on a human, the underlying principles are applicable to many different joints such as the hip, shoulder, ankle, elbow or wrist, and could also be applied to joints associated with other animals.

FIGS. 1 and 2 are provided to allow a simple explanation of certain terms that are used within this specification. FIG. 1 illustrates a standard leg having a femur 1, tibia 2 and fibula 3. These are joined at a knee joint 4. The femur defines a femoral mechanical axis 5 extending from the knee to a ball joint 6 which forms part of the person's hip. A tibial mechanical axis 7 extends from the knee 4 to the lower end 8 of the tibia itself. The femur and the lower leg (made up of the tibia 2 and fibula 3) can pivot relative to each other about a knee joint axis 9. The femur and the lower leg thus define a plane in which the respective mechanical axes pivot relative to each other. Thus, each mechanical axis will substantially align with the respective part of the leg, such that the knee joint axis 9 is perpendicular to the plane in which the axes pivot. Thus, the knee angle is thus typically the angle between the two mechanical axes. This is an idealised situation, which forms the basic geometry considered by the present invention. It is possible to apply one or more compensation schemes to deal with any misalignment between the axes and the respective part of the leg.

FIG. 2 helps to define the coordinate system associated with the knee joint, as well as how the terms pitch and roll apply to the knee. The convention when discussing the knee joint is that, when a person is standing upright, the x-axis points forward i.e. away from the knee parallel to the ground, the y-axis points to the right of the person, and the z-axis points downwards towards the ground. This convention applies to both left and right legs, i.e. the positive y-axis is always to the right hand side of the knee irrespective of the leg. In a normal knee alignment, the y-axis is therefore analogous to the knee joint axis 9.

The orientation of any sensors associated with the knee typically has two components. A rotation of the sensor about the x-axis is a roll motion, identified by arrow 18, and defines a roll angle. A rotation of the sensor about the y-axis is a pitch motion, identified by arrow 19, and defines a pitch angle.

FIG. 3 illustrates a pair of sensors 10 attached to a leg 11. Each sensor contains one or more motion sensing devices which permit either (i) the pitch and/or roll and/or yaw of the individual sensor to be determined or (ii) the relative pitch and/or roll and/or yaw between the sensors to be determined. These motion sensing devices could be any suitable devices such as, but not limited to, an accelerometer, gyroscope, or a pair of strain gauges. In other variations, more than two sensors could be used. For example, it may be desirable to use three sensors if the joint under surveillance is a ball and socket joint which has three degrees of freedom of movement. In some cases, it may be desirable to use an additional sensor on a joint such as a knee joint to assist in determining orientations of the thigh and calf. For example, two sensors could be placed on one of the user's limbs. Measurements from such a third sensor could be processed in a similar manner to the processing described above for two sensors. One possibility is to process the data from the two sensors placed on one limb to obtain a set of data for that limb, and then process that in conjunction with the data from the other limb as described above.

An upper sensor 10a is placed on the thigh 12 and a lower sensor 10b is placed on the calf 13. The purpose of the sensors is to monitor the flex of the knee at the knee joint, i.e. a pitch angle about the y-axis/knee joint axis 9. If the two sensors 10a, 10b could be aligned such that the z-axis of the sensor was parallel to the respective mechanical axis of the leg, and the sensor y-axis was parallel with the knee joint axis 9, the calculation of the knee angle would be a simple subtraction of the calf pitch angle from the thigh pitch angle.

However, as will be appreciated, the shape and form of a human leg does not generally permit such alignment to be possible, so when the sensors 10a, 10b are in place as shown in FIG. 3, there is misalignment with the femoral and tibial mechanical axes which needs to be corrected for in order to obtain an accurate knee angle measurement.

In the example of the patient having had a total knee replacement or indeed any other knee surgery or knee complaint which results in limited movement of the knee, it can be helpful for a healthcare professional, or even the patient themselves, to monitor the knee angle over lengthy period of time such as weeks or even months. Thus, a further problem can arise as the sensors will need to be removed periodically for numerous reasons including but not limited to cleaning of the sensor, recharging the sensors, increasing the comfort of the patient at night or cleaning of the patient in the sensor locations. When the sensors are removed and reapplied, however carefully this is done, there is likely to be some misalignment of the replaced sensor relative to its previous position. When this happens, the absolute value of a pitch angle or roll angle after replacement does not necessarily correlate to the absolute value of the pitch angle or roll angle prior to removal. As such, methods and/or devices which increase the accuracy of the replacement of the sensor and/or allow for some form of compensation relating to any misalignment are beneficial.

FIG. 4 illustrates a sensor mounting system by which one of the sensors 10a, 10b can be attached to the respective portion of the leg 11 in a manner which increases the accuracy of replacing the sensor after it has been removed.

The system is split into two primary parts, namely a first mount 20 shown in FIGS. 4a and 4c, and a second mount 30 shown in FIG. 4b. The first mount 20 is intended to be placed directly on to the patient and is a longer lasting part, by which we mean it is intended to be in situ for longer period of time, such as a week, than the second mount.

The second mount 30 is intended to be used to join the sensor to the first mount and is to be used for a shorter period of time, such as a day, such that the sensor can be removed for example at night to allow for recharging overnight when movement of the knee is minimal and/or more comfortable sleeping for the patient. The first and second part may be removably joined together to permit the sensor to be attached to the patient.

The first mount 20 is a patch, as shown in FIG. 4a, as being formed from a series of four layers. Other numbers of layers are possible. In FIG. 4a, the first lowermost layer 21 is the outermost layer of the patch and maybe formed from a material, such as MED 5062A, and maybe be a pliable, transparent, breathable polyethylene film with an acrylic adhesive. The transparency is beneficial as it allows for site visibility. The adhesive side of the outermost layer faces layer 22, which maybe a polyurethane film for providing strength and durability to the mount 20. The polyurethane film may be coloured to have a high contrast relative to the patient's skin to assist in aligning the sensor during reapplication. The third layer 23 is typically a double sided adhesive film, such as MED 2171 H, and preferably includes an absorbent hydrocolloid adhesive which is designed to not break down upon saturation and which provides for a low profile, assists in creating optimal skin and wound healing conditions, has a high fluid handling capacity and is breathable. The fourth layer 24 is a release layer designed to be removed so that the patch can be applied to the patient's skin. The fourth layer may preferably include a release tab 25 or other projecting feature to assist with its removal from the third layer.

Each of the first to third layers are provided with cut out portions 26 which are aligned, or at least overlap, such that once the release layer is removed, it is possible to see from the outermost first layer 21 through the skin of a patient upon whom the patch has been applied. The purpose of the cutouts is, as described later, to assist with the alignment of a replacement first patch 20 in substantially the same position as the initial patch, as the skin of the patient can be marked so that the mark(s) are visible through the patch.

The cutouts 26 may be holes through the respective layers (in which case the markings can be replenished easily by marking through the holes), or could be transparent sections within each layer. A combination of the two may be used. The cutouts 26 are shown as elongate and stadium shaped, although other shapes could be used. Whilst two cutouts are depicted in the figures, any number could be used. The number and/or shape of the cutout(s) need to assist with the alignment of a replacement first patch 20 in substantially the same position as the initial patch. As an example, a single cutout 26 may be used if the cutout is shaped to allow an orientation to be determined, eg a single irregular cross or triangle could be sufficient to determine not only position, but also orientation of the first mount 20, if a correspondingly shaped mark was on the patient's skin. The cutout may, in the plane of the layer, have one dimension significantly larger than the other to assist with providing a satisfactory tolerance for orientation.

As can be seen in FIG. 4c, the second 22 and third 23 layers are typically smaller than the first and fourth layers, such that once the release layer 24 is removed, the first layer 21 can seal onto the patient's skin around the second and third layers, i.e. totally enclosing them.

The first mount may be substantially planar, in that the thickness is significantly less than the other two dimensions. One or more of the various layers in the first mount 20 may include a waist portion 27 which is a narrowing of the layer in one of the two larger dimensions. The waist is typically located at the point at which the mount may flex and the reduced size of the waist assists in allowing this flex to happen. Additionally, the provision of the waist helps allow a use to pick up the mount from a flat surface. The first mount may be elongate in that, of the two larger dimensions, one dimension is two or more times the other dimension.

The second mount 30 or patch is shown in FIG. 4b and is a bidirectional fixing. By this, we mean that it can join two items together either by the use of a single structure having two joining surfaces, each directed towards one of the two objects to be joined, or a two part structure, each part being connected to one of the objects to be joined and having complementary features which cooperate to join the two parts together. In each case, the second mount provides fixing in two opposed directions as it must join to both the first mount and the sensor. The second mount 30 is typically slightly smaller than the footprint of the sensor so that any adhesive, if used as described below, doesn't get exposed even if the double-sided patch is not aligned very well. It also means the sensor has a free edge that is not stuck to make it easier to peel away from the leg.

In the example of FIG. 4b, the second mount comprises three layers. A main central layer 31 is a double sided adhesive layer having a pair of outer release layers 32, 33. The central layer 31 is preferably a double sided, conformable, polyester film, typically with a solventless acrylic adhesive on both sides. It may be transparent. It is preferably conformable, moisture resistant, breathable and heat sealable. The outer release layers 32, 33 may each be provided with a release tab 34 or other projecting feature to assist with its removal from the central layer.

As will be explained later, the double sided adhesive nature of the second mount or patch is used to mount the sensor to the first patch, so that a sensor can be fixed onto a patient as shown in FIG. 3.

The second mount may be substantially planar, in that the thickness is significantly less than the other two dimensions. One or more of the various layers in the second mount 30 may include a waist portion 37 which is a narrowing of the layer in one of the two larger dimensions. The waist 37 of the second mount may provide similar benefits to those provided in relation to the first mount. The second mount may be elongate in that, of the two larger dimensions, one dimension is two or more times the other dimension.

A further feature of the second mount 30 is a removal tab 35. The removal tab 35 is provided on at least the central layer and projects away from the layer, but in substantially the same plane as the layer. The tab is typically integral to the remainder of the central layer. One or both release layers 32, 33 may also have a corresponding tab. The tab 35 on the central layer is provided with cover portions 38. The cover portions are to maintain coverage of the adhesive on the central layer once the release tabs 32, 33 have been removed, so that the tab 35 can be used to assist in removal of the central layer either from a sensor to which it is applied or from the first patch 20.

In an alternative, the second mount could be formed from a two part structure such as hook and loop fasteners such as or press fit fasteners such as poppers, in which one part is fixed to the first mount, either integrally or by adhesive or the like, and another part is fixed to the sensor again either integrally or by adhesive or the like, and cooperating features such as hook and loops or press fit poppers retain the two parts together, thereby mounting the sensor to the patient. The Velcro® may be “directional” by which we mean that the hooks of the hook and loop all lie in the same direction such that the fastening system grips and holds better in one direction than the opposite direction, or even potentially only in one direction and not the opposite.

In a further alternative, a clip either on the first mount or the sensor, or a pocket on the first mount could be utilised as the second mount.

In a further alternative, one or more magnets could be utilised as the second mount.

In any of the examples, the sensor and/or the first mount may contain one or more protrusions or the like which cooperate with the other of the sensor and the first mount to assist in aligning the sensor on the first mount.

FIGS. 5 to 7 illustrate the use of first and second mounts in line with the discussion concerning FIG. 4, and therefore also a method by which the accuracy of sensor placement can be increased. These figures also illustrate a method by which any misalignment of the sensors may be compensated for by recalibration of the sensors. The compensation method may, as described herein, utilise the first and second mounts or could be carried out without the specific mounts or placement method described.

FIG. 5 illustrates a simplified version of the compensation method and will be more readily understood once the more detailed method is explained with reference to FIGS. 6 and 7.

FIG. 6a to c show how the sensors 10a, 10b are applied to a patient's leg. The leg is placed into a baseline position as shown in FIG. 6a. This is preferably a position which is easily repeatable, in particular without the use of measuring apparatus or the like, as it is a position which the patient must be able to repeat away from a medical facility, i.e. at home. Semi-permanent markings 51 are applied to the leg in the intended sensor locations. This may be done when the leg in the baseline position as shown, or may be done at an earlier stage. Preferably, a form of goniometer 50 is used so that the angle of the knee in the baseline position can be recorded. The goniometer preferably includes one or more templates 52 of the cutouts of the first mount, so that the semi-permanent markings match the cutouts. After marking, in FIG. 6b, the first mount or patch 20 can be applied by removing the release layer and then applying the first mount to the patient by aligning the cutouts 26 with the markings 51.

A second mount can then be used, typically applied to the sensor first, and then to the first mount (see FIG. 6c). The shape and/or colouring of part of the first mount can assist in aligning the sensor on the first mount.

The patient then returns their leg to the baseline position which can be checked with the goniometer if necessary and the knee angle recorded (FIG. 6e) for example by inputting data to a mobile device 55 such as a phone. The sensors however in this first position/orientation will inevitably be misaligned with the mechanical axes (femoral and tibial), and this needs to be corrected for. This can be done by calibrating this first position to enable a pitch offset to be calculated/recorded, and then applying this offset to align the difference between the pitch readings from the sensors to the knee angle (pitch) measured previously by a healthcare professional typically using the goniometer (or other suitable device). The first pitch offset is therefore the difference between the goniometer reading (knee angle) and the sensor reading. This allows the reported angle to the patient or healthcare professional to be determined in subsequent motion of the knee, as the reported angle will be the sensor reading (which is variable) plus the offset (which is now fixed). Any of the data, including pitch/roll or orientation information, offset readings, measured or reported knee angle may be stored on one or more of the sensors and/or maybe input into any form of computer type device, such as desktop computer, a mobile phone, a tablet or a laptop. The input may be carried out by automatically transmission of the data from one of the sensors and may be either contemporaneous, i.e. streamed for use in real time, or may be sent only periodically.

Typically, the first mount/patch will be in place for a week before it requires removal to allow for cleaning of the site of the sensor. However, on a shorter timescale, e.g at the end of a day, the sensor will need to be removed along with the second mount (or part of the second mount if a two part mount is used) for any of the reasons previously explained. When replaced, the sensor 10a, 10b may or may not be replaced in exactly the same position as previously and so the sensors have a second position. As such, before further useful readings can be taken, the patient must place their leg back into the baseline position, but without the benefit of a goniometer or the like (which is why an easy to repeat position is preferred). The sensors must then be recalibrated in the same manner as above to provide a second pitch offset (the difference between the knee angle from the initial set up and the sensor reading taken in the second position). For motion of the knee subsequent to the replacement, the reported angle is the sensor reading plus the second pitch offset. It is preferable that the system for recording data about the patient's movement will not record new data until the offset has been updated.

FIG. 7 shows how the first mount or patch can be replaced. Initially at FIG. 7a, the markings 51 on the leg 11 are replenished to ensure they can be seen clearly. The first mount 20 is then removed (FIG. 7b) to allow for cleaning and or hair removal in the area around the markings 51 (FIG. 7c). The new first mount 20 can then be applied (FIG. 7d) using the cutouts 26 in the first mount and the visual markings 51 as a guide. Pressure can then be applied (FIG. 7e) to ensure the first mount 20 is securely fixed in place.

FIG. 5 sets out the broad methodology associated with the compensation/calibration of the sensors. At step 51, the joint to be monitored, and therefore the one about which two sensors have been placed, one on each side of the joint, is placed into the baseline position. This baseline position is that shown in FIG. 6d.

As described previously, it is beneficial to monitor movement of a knee after total knee replacement and in this situation, a patient is typically able to straighten their leg, but will struggle to bend it, such that the sensors are beneficial to track the patient's movements and hopeful improvement in movement over an extended period of time such as weeks or months. Thus, the preferred position is a “limit of movement position”, and in relation to a knee joint, this is a passive full extension position. This is, in effect, the position the leg takes up when extended along a horizontal surface.

Step 52 is a calibration of the sensors to the baseline position, whatever that knee angle might be. This calibration allows the sensors to set the first orientation (pitch and/or roll) in which they are placed as equivalent to the baseline position. Any motion of the leg, and therefore the sensor, relative to that calibrated first orientation can then be understood.

As has been described, the sensors are removable for numerous reasons. Whilst the method described in relation to FIGS. 6 and 7 helps to reduce misalignment, it does not necessarily prevent it happening, so the sensors may be replaced in a different second orientation. In order for the data generated by the sensor after replacement of the sensor to be analogous to the data before replacement, any difference in orientation needs to be recognised. Thus, after the sensor has been removed and replaced at step 53, the joint being monitored needs to be placed back into the baseline position as in step 54. This may include the use of a control system which only permits further data to be recorded and/or stored once the recalibration has been done. The control system may be on one or more of the sensors themselves or may be located remote from the sensors. The sensors can then be recalibrated at step 55 such that any offset in pitch and/or roll angle of the sensor relative to the initial readings can be adjusted for. The initial readings of the baseline position may also be updated, for example by the healthcare professional, as it is possible for the baseline position to change over time. This is particularly true in the period immediately after surgery, when a patient is seeing a healthcare professional more regularly. Immediately after surgery, a patient may be unable to fully extend the knee, but after one or more weeks may find that the they can do so. As such, the baseline position would have changed, so the initial readings will need to be updated.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A method of calibrating a first sensor and a second sensor mounted to a joint of a patient, the method comprising the steps of:
(a) in a baseline position of the joint to be measured in which the first sensor and the second sensor are in a first configuration relative to each other, determining a first offset between a measured joint angle and an angle between the first sensor and the second sensor in the first configuration, for calibrating at least one of the first sensor and the second sensor, the angle between the first sensor and the second sensor in the first configuration corresponds to a difference between at least one of a pitch angle and a roll angle of the first sensor and at least one of a pitch angle and a roll angle of the second sensor;
(b) after at least one of the first sensor and the second sensor has been removed and reapplied, placing the joint back into the baseline position such that the first sensor and the second sensor are in a second configuration relative to each other; and
(c) determining a second offset between the measured joint angle and an angle between the first sensor and the second sensor in the second configuration for recalibrating at least one of the first sensor and the second sensor such that, in each of the first configuration and the second configuration, a same joint angle for the baseline position is reported, the angle between the first sensor and the second sensor in the second configuration corresponds to the difference between at least one of the pitch angle and the roll angle of the first sensor and at least one of the pitch angle and the roll angle of the second sensor.

2. The method according to claim 1, wherein the first sensor and the second sensor communicate such that the angle between the first sensor and the second sensor is determined by one of the first sensor or the second sensor.

3. The method according to claim 1, further comprising, prior to (a), the step of measuring the joint angle by using a goniometer.

4. The method according to claim 1, wherein recalibrating the at least one of the first sensor and the second sensor is carried out as part of a sensor activation process.

5. The method according to claim 1, wherein measuring the baseline position includes measuring the joint angle between the respective portions of the joint.

6. The method according to claim 5, wherein the measured joint angle is a pitch angle and/or a roll angle.

7. The method according to claim 1, further comprising the step of moving the joint to the baseline position which is preferably a joint full extension position.

8. The method according to claim 1, wherein reapplication of the at least one of the first sensor and the second sensor is carried out at substantially a same position.

9. The method according to claim 1, further comprising the step of identifying an axis of movement of the joint.

10. The method according to claim 1, further comprising the step of applying the first sensor and the second sensor, one on each side of the joint.

11. The method according to claim 10, further comprising the step of, prior to applying the first sensor and the second sensor, marking locations for mounting at least one of the first sensor and the second sensor on each side of the joint.

12. A system for recording angular position changes in a joint, the system comprising:
a pair of sensors, each sensor being placed, in use, on a respective side of the joint, each sensor of the pair of sensors including a data transmission device for providing data relating to an orientation of each sensor;
a data storage device for receiving data from one or more of the sensors, the data relating to the orientation of one or both sensors; and
a control system configured to recognize when one of the pair of sensors has been removed from the joint and to require a recalibration of alignment of the sensors prior to recording a subsequent data set.

13-25. (canceled)

26. The method of claim 1 further comprising prior to (b) calibrating the at least one of the first sensor and the second sensor based on the first offset.

27. The method of claim 26, wherein calibrating the at least one of the first sensor and the second sensor includes applying the first offset to align the difference between the angle between the first sensor and the second sensor in the first configuration to the measured joint angle.

28. The method of claim 1, further comprising after (d) recalibrating at least one of the first sensor and the second sensor such that, in each of the first configuration and the second configuration, the same joint angle for the baseline position is reported.

29. The method of claim 28, wherein recalibrating the at least one of the first sensor and the second sensor includes applying the second offset to align the difference between the angle between the first sensor and the second sensor in the second configuration to the measured joint angle.

30. The method according to claim 1, wherein the first sensor is configured to mount to a first mounting system defining at least one first cutout portion, the second sensor configured to mount to a second mounting system defining at least one second cutout portion, the method further comprising prior to (a):
applying the first mounting system to a first side of the joint and the second mounting system to a second side of the joint;
marking a first location of the at least one first cutout portion and a second location of the at least one second cutout portion; and
attaching the first sensor to the first mounting system and the second sensor to the second mounting system so that the first sensor and the second sensor are in the first configuration relative to each other.

31. The method of claim 30, further comprising prior to (b) reapplying the first sensor and the second sensor.

32. The method of claim 31, wherein reapplying the first sensor and the second sensor includes aligning the at least one first cutout portion of the first mounting system with the first location and aligning the at least one second cutout portion of the second mounting system with the second location.

33. The method of claim 32, wherein reapplying the first sensor and the second sensor includes reattaching at least one of the first sensor to the first mounting system and the second sensor to the second mounting system.
